(19) United States
(12) Patent Application Publication
(10) Pub. No.: US 2006/0254371 A1
(43) Pub. Date: Nov. 16, 2006

(54) Apparatus and method for collecting soil solution samples

(76) Inventors: Amnon Shiloni, Shoham (IL); Yoram Kadman, Negev (IL)

Correspondence Address: DENNISON, SCHULTZ & MACDONALD, 1727 KING STREET, SUITE 105, ALEXANDRIA, VA, 22314, US

(21) Appl. No.: 10/545,163

(22) PCT Filed: Feb. 16, 2004
(86) PCT No.: PCT/IL04/00130

§ 371 Date: Aug. 11, 2005

(30) Foreign Application Priority Data
Feb. 11, 2003 (IL) 154387

(51) Int. Cl. G01N 1/14; G01N 35/10; G01N 33/24

(52) U.S. Cl. 73/864.34; 73/863.01; 73/864.85

(57) ABSTRACT

A system for collecting soil solution samples including a sampling device arranged to contact soil and coupled to a vacuum source, and a suction unit for automatically controlling pressure and time of operation of the vacuum source to provide suction for withdrawing solution from the soil through the sampling device and abrupt release of the vacuum at the end of a pre-defined sampling period, and a method for collecting soil solution samples including coupling a sampling device to a vacuum source, planting the sampling device in soil to be monitored, automatically controlling pressure and time of operation of the vacuum source to provide suction for withdrawing solution from the soil through said sampling device, and automatically stopping the vacuum source and abruptly releasing the vacuum at the end of a pre-defined sampling period.

### FIELD OF THE INVENTION

The present invention relates to devices for collecting soil solution samples in general and, in particular, to a device for sampling soil solutions, in order to optimize irrigation and fertilization.

### BACKGROUND OF THE INVENTION

Over the past few decades, agricultural technology has developed by leaps and bounds. One of the most important advances in the field concerns fertilizer irrigation control (fertigation). Based on real time samples of soil solutions, this technology is geared towards improving the yield and quality of the final product, as well as saving water and fertilizer costs.

The goals of fertigation control are:
To improve the efficacy of water use and fertilizers.
To save costs.
To avoid mistakes in the growing process.
To keep the environment under control (e.g., avoid contamination of underground reservoirs).

The existing technology is principally manual, involving simple instruments and a great deal of manpower. Unfortunately, the process itself is too complicated for most farmers. Thus, although the benefits of manual real time control (RTC) of fertigation for fine-tuning the fertilization and irrigation process of various crops are common knowledge, few farmers routinely use the technique.

In order to utilize RTC technology, farmers or crop growers manually extract soil solution samples adjacent the roots of the plants by means of a syringe inserted into a soil solution extraction tube planted in the ground adjacent the roots. Typically, extraction tubes for sampling are installed at at least two different depths in every sampling site, according to the type of plant and the typical root zone shape. For instance, in order to take samples at three different depths, the root zone space is divided into three layers: one-third from the soil surface, two-thirds from the soil surface, and at the bottom of the root zone. An extraction tube is installed in the soil solution at each layer.

The sampling process begins some time after irrigation is complete, with the delay ranging from minutes to hours, in order to carry out the sampling in optimal conditions, preferably when the soil solution state is stable. The two parameters which have the greatest effect on the water distribution in the irrigated soil are soil type (light, medium or heavy) and the system used in irrigation (drip, sprinkling or furrow). Vacuum suction of the solutions from the soil generally takes place over several hours, depending on the type of soil. The vacuum power applied by the farmer using the syringe, time delay from end of irrigation, and the amount of time suction is applied until sampling is completed naturally differ each time a sample is extracted. In particular, the extraction tube slowly returns to atmospheric pressure over time, when vacuum is no longer applied, so there is no abrupt release of vacuum and there can be no precise calculation of the volume collected or sampling time. This is problematic, as the process results in inconsistent conditions and results. When the fine tuning of the system is more accurate, more significant results can be achieved.

When the sampling process is complete, the soil solution samples are collected by the farmer from the fields and taken to a lab to be analyzed. Alternatively, the samples can be analyzed at the site of sampling using a field test kit. The lab results are examined by the grower who, by following the changes in each tested parameter, such as salinity (e.c.), acidity (pH), and NPK, over time, is now capable of making educated decisions about irrigation and fertilization, so as to regulate irrigation and fertilizer use more accurately and efficiently. For example, using the accumulated data, the farmer can select sampling conditions (amount of time after irrigation is complete, time span of the sampling and the vacuum power in the system), and fertigation parameters (water quantities and fertilization solution content).

As things stand today, the sampling process is complicated and the results are inadequate. Farmers must be present at each sampling station at the beginning of the process to operate the measuring equipment, and either have to wait until the vacuum suction is complete, or must rush back immediately after its conclusion, in order to release the pressure in the soil extraction tubes so as to achieve close to controlled conditions. Because this requires extensive mental and physical activity above and beyond the actual tending of the plants and the results are inconsistent, not many farmers use this manual technology. This holds true even for farmers aware of its immense economic and environmental benefits.

In addition, when using the manual technique, and because results garnered from the manual process are not completely accurate, the solution extraction is forced to depend on all kinds of environmental conditions, such as precipitation, and temperature. Thus, neither the sampling conditions nor the results are precise.

There are also known suction lysimeters, as described, for example in U.S. Pat. No. 6,609,434. These lysimeters are inserted in a bore in the soil to be sampled, vacuum is applied to suck soil solution into the lysimeter, and then the lysimeter is removed from the bore and the sample is analyzed.

Devices for analyzing collected soil solution samples are also well known. These generally sit in a laboratory to which the samples are brought for analysis. There are also known various sensors for measuring various parameters, which are inserted directly into the soil to be tested in the field.

Accordingly, there is a long felt need for a simple device for taking soil solution samples under precisely controlled conditions for determination of fertigation parameters, and it would be very desirable if such device were substantially automatic.

### SUMMARY OF THE INVENTION

The present invention provides a system and method for automatically collecting soil solution samples at predetermined intervals after irrigation, and for a predetermined and controlled sampling time.

According to the present invention, there is provided a system for collecting soil solution samples including a sampling device arranged to be planted in soil to be monitored, the sampling device being coupled to a vacuum source, and a suction unit for automatically controlling pressure and time of operation of the vacuum source to provide suction for withdrawing solution from the soil through the sampling device, the suction unit including a relief valve for abrupt release of vacuum at the end of a pre-defined sampling period.

According to one embodiment of the invention, the sampling device includes a porous extraction tube arranged to be planted in the soil, and a solution collecting bottle coupled to the extraction tube, the collecting bottle being coupled to the vacuum source.

According to one embodiment of the invention, there is provided a system for collecting soil solution samples including a porous soil solution extraction tube arranged to be implanted in soil, a solution collecting bottle coupled to the soil solution extraction tube by a vacuum tube, and a suction unit including a vacuum pump coupled to the solution collecting bottle for automatically controlling pressure and time of operation of the vacuum pump to provide suction for withdrawing solution from the soil through the extraction tube and into the solution collecting bottle, the suction unit including a relief valve for abrupt release of vacuum at the end of a pre-defined sampling period.

Further according to one embodiment, the system for taking soil solution samples includes a timer adapted to begin counting a pre-selected delay time at the end of irrigation, the timer being coupled to a vacuum pump coupled to sampling devices inserted at desired locations in the soil, for automatically sucking solution from the soil adjacent the sampling devices at the end of the delay time, and means for automatically releasing the vacuum after a pre-selected sampling period.

According to a preferred embodiment of the invention, the system further includes a plurality of testing sensors mountable in each collecting bottle, the sensors being coupled to an external location, such as a central computer or a data log, for transferring electrical signals corresponding to data collected by the sensors for analysis.

Further according to the present invention, there is provided a method for collecting soil solution samples including coupling a sampling device to a vacuum source, planting the sampling device in soil, automatically controlling pressure and time of operation of the vacuum source to provide suction for withdrawing solution from the soil through the sampling device, and automatically stopping the vacuum source and abruptly releasing vacuum at the end of a pre-defined sampling period.

There is also provided, according to the invention, a method for collecting soil solution samples including implanting at least one soil solution extraction tube in soil, coupling a solution collecting bottle to the soil solution, extraction tube by a vacuum tube, coupling a suction unit including a vacuum pump to the solution collecting bottle, automatically controlling pressure and time of operation of the vacuum pump to provide suction for withdrawing solution from the soil through the extraction tube and into the solution collecting bottle, and automatically stopping the vacuum pump and abruptly releasing vacuum at the end of a pre-defined sampling period.

According to one embodiment, the step of automatically controlling includes activating a timer at termination of irrigation to begin counting a pre-selected delay time, causing the timer to automatically initiate vacuum to suck solution from soil adjacent the sampling tubes into the sampling tubes at the end of the delay time, and automatically stopping the vacuum and releasing the vacuum after a pre-selected sampling period.

### BRIEF DESCRIPTION OF THE DRAWINGS

The present invention will be further understood and appreciated from the following detailed description taken in conjunction with the drawings in which:

FIG. 1 is a schematic illustration of a single unit sampling station in a system for collecting soil solution samples constructed and operative in accordance with one embodiment of the invention;

FIG. 2 is a schematic illustration of a multiple unit sampling station constructed and operative in accordance with one embodiment of the invention;

FIG. 3 is a schematic illustration of a field position scheme of a system for collecting soil solution samples including an operating control unit and a plurality of sampling stations, according to one embodiment of the invention;

FIG. 4 is a flow chart illustrating the method of operation of one embodiment of the present invention;

FIG. 5 is a schematic illustration of a suction unit according to one embodiment of the invention;

FIG. 6 is a schematic illustration of a system for collecting soil solution samples constructed and operative in accordance with a further embodiment of the invention, including transferring the soil solution samples to a lab station; and

FIG. 7 is a schematic illustration of a solution collecting bottle for use in a system for collecting soil solution samples constructed and operative in accordance with yet another embodiment of the invention, including real-time analysis.

### DETAILED DESCRIPTION OF THE INVENTION

The present invention relates to an automatic control system for collecting soil solution samples that can closely control the extraction of soil solution on a real time basis. The system of the present invention controls the following main parameters in the solution sampling process:
A. The length of time a farmer waits between the completion of irrigation and initiation of sampling.
B. The length of time of extraction (sampling), or the volume of extracted solution, for each sampling.
C. The vacuum power for extraction (which typically depends on the soil type).

Each parameter is influenced by the type of soil, the system used in irrigation, and the condition of the soil before irrigation. While the parameters cannot be fixed absolutely, farmers will be able to approach optimum conditions. Once the preferred parameters have been determined, the system may utilize substantially identical parameters for each sampling. On the other hand, it will be appreciated that different sampling locations may have different optimum parameters.

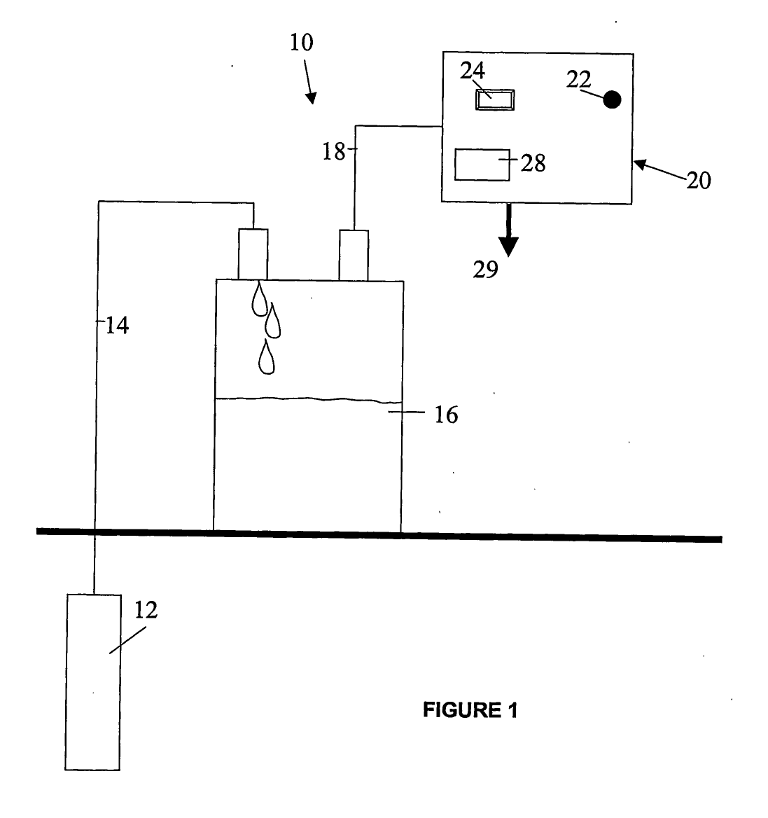

Referring now to FIG. 1, there is shown a schematic illustration of a single unit sampling station 10 in a system for collecting soil solution samples constructed and operative in accordance with one embodiment of the invention. Sampling station 10 includes an extraction tube 12 implanted in the soil in the location to be monitored. Sampling device 12 can be, for example, a porous clay extraction tube, as known in the art. Extraction tube 12 is coupled by collecting vacuum tubing 14 to a solution collecting bottle 16. For purposes of the present invention, a solution collecting bottle refers to any container of suitable size and shape for collecting the desired volume of solution extracted from the soil, and can be as small as a test tube. Solution collecting bottle 16, in turn, is coupled via control vacuum tubing 18 to a vacuum pump in a suction unit 20. Suction unit 20 may be controlled by a mechanical or electronic timer or counter, as described below, thus creating an automatic electromechanical suction system.

The timing of the start of sampling can be selected in a number of ways. At present, the following ways are contemplated. The timing of the sampling cycle can be selected by the farmer, based on professional guidelines and suggestions. Activation of the pump or the timer can be manual, but coordinated with the starting or finishing of irrigation of the monitored location. Alternatively, activation of the pump, or of the timer can be automatically synchronized with the irrigation controller. Or sampling can begin automatically upon activation by a sensor in the soil, for example, a soil moisture sensor, such as a tensiometer.

In the illustrated embodiment, suction unit 20 includes a timer (not shown). When the timer receives an activation signal, it begins to count a pre-set delay time to start the pump for sampling. Once the pump is started, the timer counts a pre-set sampling period and stops the pump when the sampling period expires. Suction unit 20 also includes a manual operating switch 24, for manual activation of sampling, without a delay time. The sampling period is automatically ended by the timer after the expiration of the pre-set sampling period, preferably by opening a relief valve to abruptly pressurize the tubing. This provides a precisely defined and easily repeatable sampling time, which is virtually impossible with conventional systems.

According to one embodiment of the invention, the timer is coupled to a reset button 22. When a sampling cycle is finished, the timer waits in a standby mode to receive a reset signal. The reset signal initiates a new sampling cycle (so that every irrigation cycle does not automatically result in sampling).

After reset, the timer waits for an activation signal, at which time the timer is initialized and starts counting the sampling cycle. The reset button 22 may be pressed manually, or the timer may be reset by a remote activation means, such as a standard remote control, mobile phone, radio signal, through the irrigation controller itself, or by any other means. Optionally, suction unit 20 may include an LED or other indicator, which is activated when the system is working (i.e., when the system is under vacuum for taking soil solution samples), and possibly another indicator to show when the timer has been reset, and the system is awaiting an activation signal.

Suction unit 20 further includes a vacuum pump 28, preferably an electric pump, or other source of vacuum. After the pre-set delay period from activation of the timer has elapsed, or upon activation of the manual or automatic activation switch, vacuum pump 28 begins to reduce pressure in the vacuum tubing 14 and 18, thereby drawing solution from the soil, through the solution extraction tube, into the soil solution collecting bottle by suction. Sampling continues for a pre-selected period of time, as counted by the timer, and then the vacuum is released and vacuum pump is turned off. Preferably, a pressure relief valve 29 is provided, which is opened after the sampling period expires, to release the vacuum abruptly.

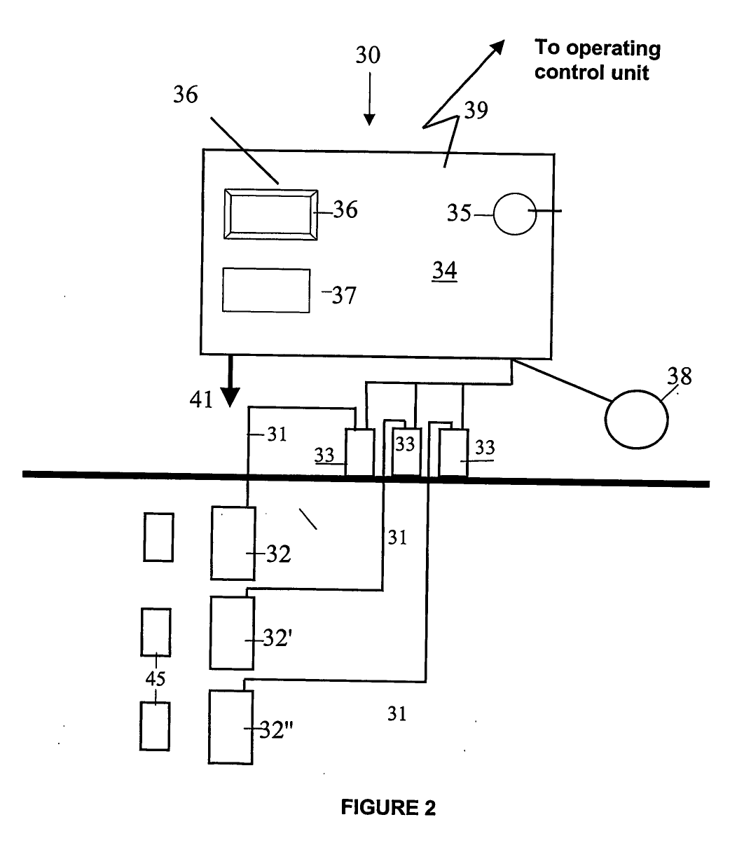

FIG. 2 is a schematic illustration of a multiple unit sampling station 30 in a system for collecting soil solution samples constructed and operative in accordance with one embodiment of the invention. This embodiment is ideal for providing indications of the soil solution contents at various depths adjacent the roots of a plant. Sampling station 30 includes three extraction tubes 32, 32′ and 32″ implanted at different depths in the location to be monitored. It will be appreciated that, alternatively, a sampling station can include one, two, or more extraction tubes, as desired for that particular location.

Each extraction tube 32, 32′, 32″ is coupled by collecting vacuum tubing 31, 31′, 31″ to a solution collecting bottle 33, 33′, 33″ and via control vacuum tubing to a suction unit 34, similar to that shown in FIG. 1. Suction unit 34 controls the sampling conditions, time and process for all three extraction tubes, and includes a manual reset switch 35, a manual activation switch 36, a vacuum pump 37 and a vacuum relief valve 41, substantially as described above. According to one embodiment of the invention, all three samples are taken at the same time, in parallel. Alternatively, sampling can be performed in the different extraction tubes at different times (i.e., after different delays), for example, each depth at a different time. In addition, suction unit 34 preferably includes means permitting remote activation of the suction unit, i.e., for resetting and/or activating the timer. Alternatively, or in addition, a sensor, for sensing a condition of the soil which determines the proper time to start the sampling period, may be provided in the soil for automatic activation of the timer and thus, the pump. For example, a soil moisture sensor 45 may be provided adjacent each extraction tube 32 to sense the moisture level in the soil at the sampling location. Soil moisture sensor 45 can be coupled by wire or wireless communications, as known, to suction unit 34 for activating the timer by means of a remote activating input signal, when the moisture level reaches a pre-selected value. Should additional information be desired, it is also possible to mount a camera (not shown), such as a video camera, at the sampling site, to supply photographs in real time, and to collect further data.

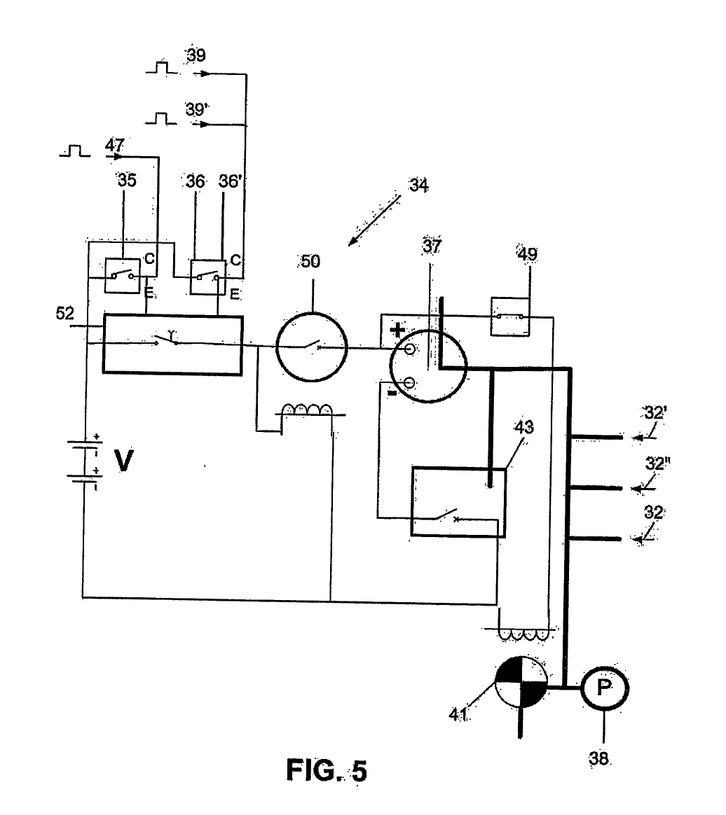

FIG. 5 is a diagram showing schematically the various components of suction unit 34. The dark lines indicate pressurized tubing, while the lighter lines indicate electrical wiring. The circuit is powered by a voltage source V, which can be the irrigation controller current, batteries in the soil sampling system control box, a solar panel, or any other external power source. The suction unit 34 includes pump 37, powered by voltage source V and controlled by an electric timer switch 52 and a relay switch 50, connected in series. Timer switch 52 may be installed either in the main irrigation controller box or, as shown here, inside the suction unit of the soil sampling system.

Timer switch 52 can receive control signals from an activation button 36 or a reset button 35. Activation button 36 can be actuated manually by a farmer by physically pressing the button. This closes timer switch 52 and begins the counting of the delay period. Alternatively, activation button 36 may be actuated remotely, as described above, by means of a remote signal 39. If it is desired to cause the timer to immediately activate the vacuum pump and begin counting the sampling period (without a delay period), a different activation button 36′ or activation signal 39′ may be provided.

According to one embodiment of the invention, a reset button 35 is also provided, in order to prevent sampling from occurring after each irrigation cycle. Reset button 35 can be actuated manually by a farmer by physically pressing the button. This closes timer switch 52 and causes the timer to stop counting and await an activation signal. Alternatively, reset button 35 may be actuated remotely, as described above, by means of a remote signal 47.

It is a particular feature of the present invention that the pressure (vacuum) is carefully controlled at all times. Thus, suction unit 34 includes a pressostat (vacuum switch) 43 or other pressure controller, which also controls pump 37. Pressure controller 43 maintains the working pressure of the system, which changes according to the conditions at the sampling location. Thus, pump 37 is de-activated and re-activated, by pressure controller 43, depending upon the pressure in the tubing. When pressure is, lower than the desired level of vacuum, the pressostat will open, thereby stopping operation of the pump. It will be appreciated that while the pump in stopped in this fashion, the pressure in the system gradually increases, since the relief valve is not opened. As soon as the vacuum returns to the desired level, the pressostat closes, thus current is allowed to flow and re-activate the pump.

An external pressure gauge 38 may also be provided to indicate the actual pressure in the vacuum tubing and the system, thereby permitting a user to monitor the vacuum pressure in the tubing and, if necessary, manually operate the vacuum relief valve by means of a manual valve switch 49. Pressostat 33 may be adjustable, thus providing for adjusting the vacuum to a desired level. In addition, since the vacuum gauge is preferably absolutely calibrated, it can be used for calibrating the system, as its readings can be relied upon while calibrating the pressostat.

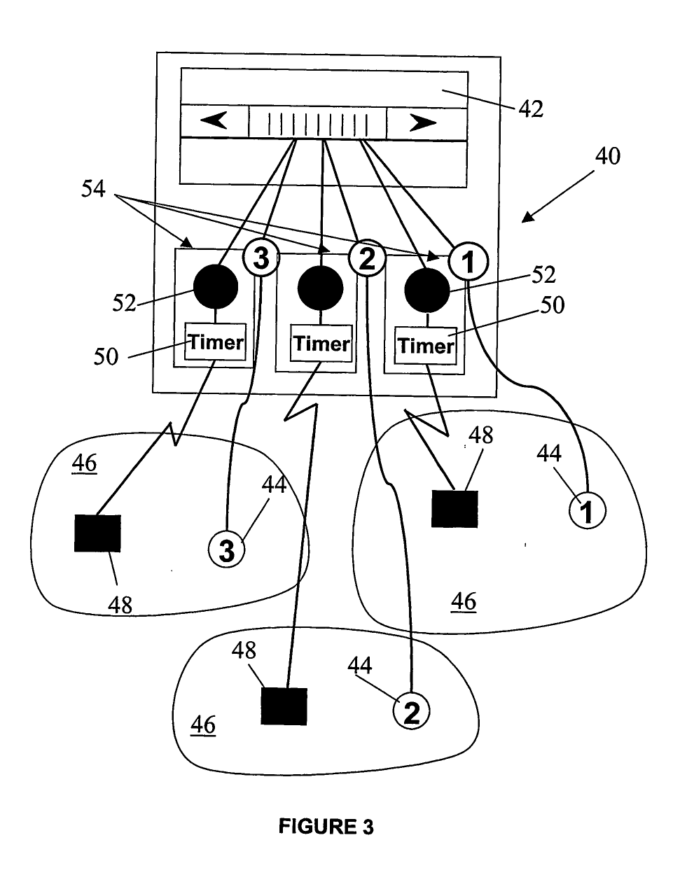

One example of an apparatus 40 for collecting soil solution samples according to the invention is shown schematically in FIG. 3, in a field position scheme. Apparatus 40 includes an irrigation controller 42, which can be a conventional computerized controller, in an operation control unit, which can be located in a central or easily accessible location. Irrigation controller 42 is coupled to a plurality of irrigation valves 44, each located in a different field or irrigation plot, by a hydraulic tube or electric cable, as known. In the illustrated embodiment, each irrigation valve is located in its own irrigation plot 46.

Each irrigation plot 46 also includes a sampling station 48 according to the present invention. The embodiment of FIG. 3 further includes a plurality of suction units 54, substantially as described above, located in the operation control unit, each associated with a different sampling station. The operation control unit also houses a vacuum pump (not shown). Each of suction units 54 includes a timer 58 or other means of actuating the relay switch to activate the vacuum pump. A reset switch 59 is provided in the operation control unit between the irrigation controller 42 and each timer 58.

The timer receives an actuating signal, preferably an output signal from the irrigation controller or from the suction unit of each sampling station. Once irrigation ends, the timer is automatically activated by the signal coming from the irrigation controller and starts counting time (begins its cycle).

The timer's active cycle includes:
A. Counting the delay period from the end of irrigation to the start of the sampling.
B. Starting the system sampling (closing the relay switch, so the vacuum pump begins to operate).
C. Counting the preset sampling period (at the end of which, the relief valve is opened and the pump is turned off).

When the sampling process is terminated, the suction unit goes into a standby mode until the next reset signal is received by the timer.

According to one embodiment, the timer will not start working again until the “reset” button is pressed. According to this embodiment, the sampling system will not work if the switch is not manually reset to “on”, even though the irrigation controller will send the activation signal to the timer. It will be appreciated that, after completion of the sampling cycle, the farmer must empty the solution collecting bottles, before activating the Reset button for the next cycle. It may not be necessary to take soil solution samples after every fertigation cycle. Rather, the farmer decides, according to local conditions, when sampling is to occur, and sets the Reset button accordingly, only after those irrigation cycles which were selected for sampling. It will further be appreciated that setting of the Reset button can be accomplished manually, or by means of a remote control system. As stated above, the controller can receive a “reset” order from one of the following options: standard remote control, mobile phone, SMS signal, through the irrigation controller itself, or by other means.

The following parameters are preferably controlled by the timer:
1. Delay Function—fixing the time delay from the termination of irrigation to the activation of the vacuum pump (vacuum suction in the soil).
2. Sampling Time function: fixing the time period for sampling (while the vacuum pump is switched on). This function will be fixed manually for a range of between one and 24 hours.

These parameters can be fixed either by a digital system—controlled through remote control—or manually. The length of time from the end of irrigation to the start of the sampling can be manually adjustable according to environmental conditions, i.e., recent rain or drought, etc. Similarly, manual adjustment of sampling time is possible. Preferably, the timer itself is controlled by the “Reset” switch, may also initialize the counter. The timer will be equipped with a mini lamp showing whether it is in the “standby” or “active” position (preferably, an LED).

According to another embodiment, the sampling can be started by an output signal from a remote device operated by the farmer, or a soil moisture sensor planted in the field adjacent the soil sampling station, or other external component of the system. Furthermore, the sampling period (i.e., the time until the relay switch is opened) can be fixed by the volume of solution collected in the solution collecting bottle, rather than a fixed length of time. (It will be appreciated that the rate of filling the solution collecting bottle can be an important parameter concerning the state of the soil adjacent the extraction tube.) Immediate release of the vacuum at the end of the sampling period helps to achieve the maximum controlled conditions, which provides better over-all results, and is provided at the end of the sampling session, which is pre-defined, as by length of time or quantity of solution collected.

It will be appreciated that the timing of the start of sampling need not be identical for all the sampling stations. Thus, different timing can be provided, for example, for sampling stations at different depths in the field, or for sampling stations in different areas of the location to be monitored, and can be triggered and counted in any of the ways mentioned above, or in any other fashion.

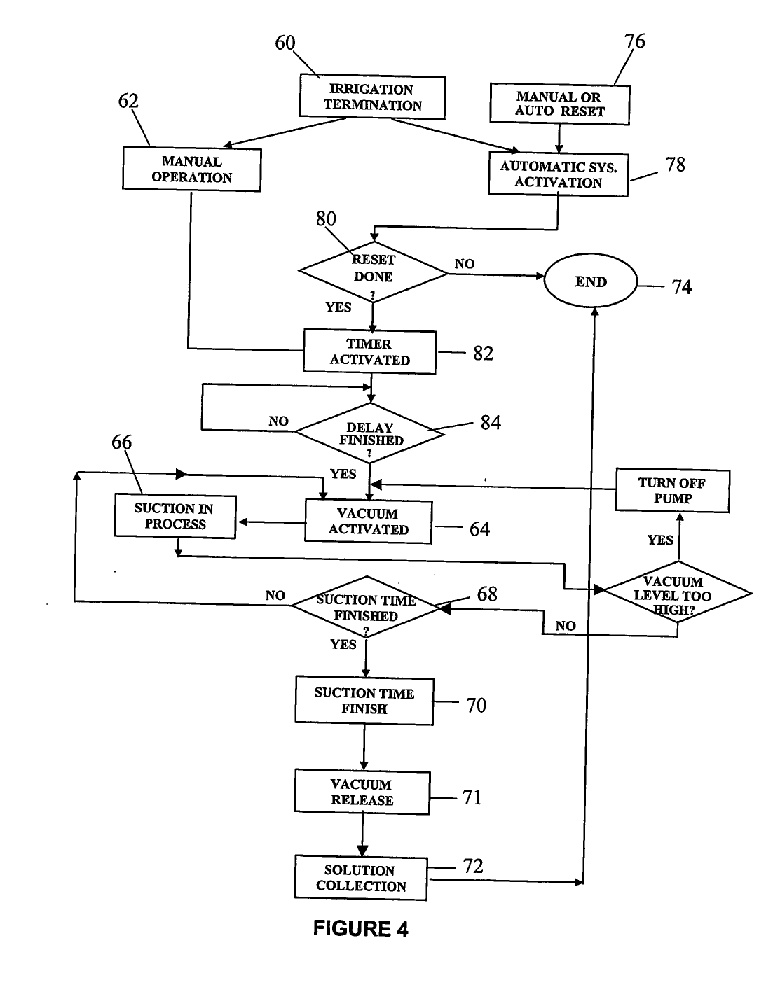

Operation of these embodiments of the system of the present invention is as follows, as illustrated schematically in FIG. 4. The cycle begins upon termination of irrigation (block 60). For manual operation, a farmer waits a selected amount of time, and the presses the manual activation button (block 62). The manual activation button serves to activate the timer (block 82) without delay (i.e., delay is finished (block 84)) which activates the vacuum (block 64). Suction is now provided (block 66), thereby drawing solution from the soil, through the extraction tube and into the collecting bottle. The pressostat continuously determines whether the vacuum level is too high (block 65). If it is, the pressostat turns the pump off (block 67) until the vacuum level is correct, at which time, the vacuum pump is activated again (block 64). If the vacuum level is not too high, the suction continues (block 68) until the pre-selected suction time (sampling period) has passed (block 70), or the pre-selected volume of collected solution has been obtained, or the end of the sampling cycle has been reached, as determined by another pre-defined parameter, at which time the vacuum is released (block 71), thereby stopping the suction of solution into the collecting bottles. The solutions from the collecting bottles are collected by the farmer (block 72) and the cycle ends (block 74). The collected solutions are now taken to a lab for analysis of the various soil parameters in each sampling location.

For automatic operation with a timer, the cycle begins with reset of the timer, which can be manual or automatic (block 76). Upon termination of irrigation (block 60), the timer receives an activation signal (block 78). The system determines whether the timer has been reset (block 80). If not, the cycle ends (block 74). If there has been reset, the timer is activated (block 82) to begin the pre-selected time delay. Once the time delay has finished (block 84), the timer activates the vacuum (block 64), and the process continues as described above with regard to the manual procedure.

It is a particular feature of the invention that, when the pre-set sampling period has elapsed, timer 52 opens relay switch 50 to stop operation of the pump and simultaneously actuates the relief valve 41 for substantially instantaneous pressurization of the tubing system. At this time, the timer goes into the standby mode, until it receives a reset signal.

One of the onerous tasks of the conventional soil solution sampling systems is the transport of filled sampling bottles to a lab for analysis or, at the very least, sending someone with a field test kit to the site to analyze the solution samples. The lab analysis preferably determines the following parameters:
A. Soil solution salinity (e.c.).
B. Soil solution acidity (pH).
C. Concentration and availability of nitrogen, potassium and phosphorus (NPK), or any other element required by the particular plant.

The NPK are macro elements, which are essential to the plant and are consumed in significant amounts during the growing period. The amount consumed changes according to the growing conditions and the stage of plant growth. Fine tuning of the quantities of each of the NPK elements is very important to achieve the best growth. Analysis of the lab results will be the basis for the farmer in making final decisions about fertilizer, irrigation and other required treatment of the soil in each sampling location. Preferably the amount of solution or liquid volume collected in each sampling bottle over a given sampling time should also be measured and recorded, as this parameter can be an important indicator of various characteristics of the soil at a given time.

According to the following embodiments of the invention, the sample transport process, too, can be automated, so as to increase the consistency of the sampling procedure and improve results.

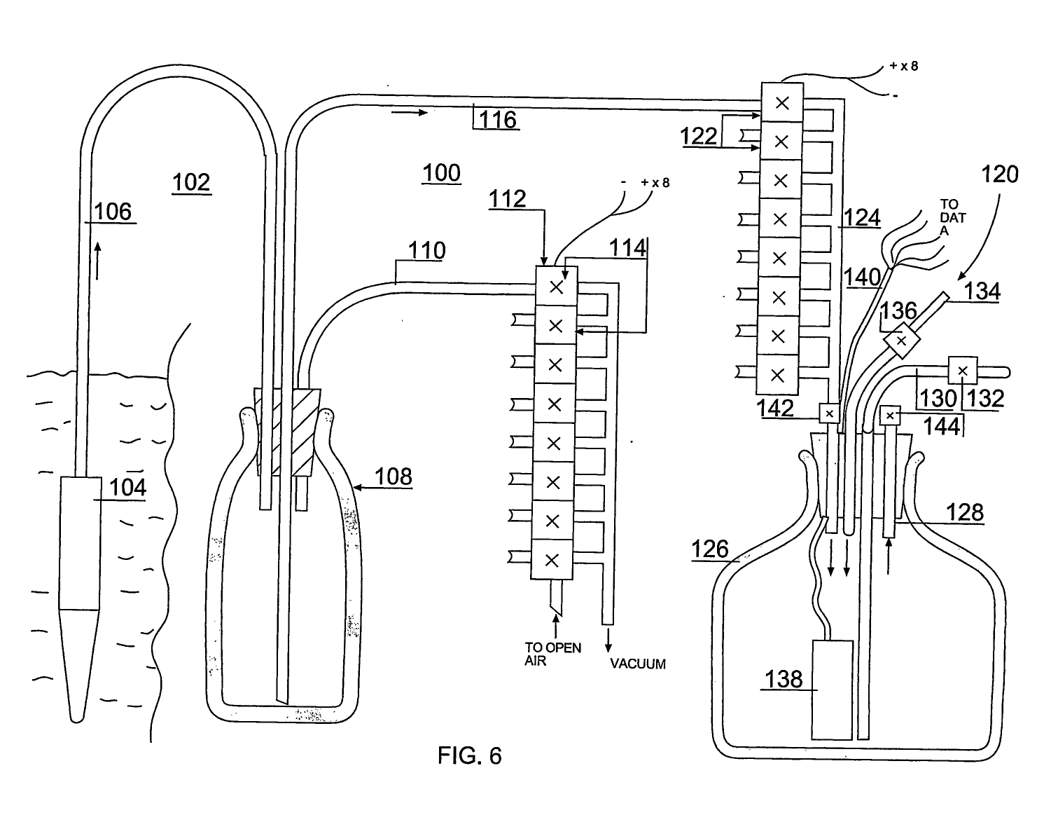

Referring now to FIG. 6, there is shown a schematic illustration of a system 100 for collecting soil solution samples constructed and operative in accordance with a further embodiment of the invention. This embodiment does not require the farmer to transport solution collecting bottles to a lab, but rather includes means for automatically transferring the soil solution samples to a lab station. It will be appreciated, in this embodiment, the timer can be a part of a controller, such as a pc computer, and the controller can be programmed to the different needs of the different sampling stations. The controller would include a program for the timer for each station, or group of stations which can be simultaneously sampled. Alternatively, this control can be carried out by an irrigation controller, which must switch between several programs for the timer for different groups of sampling stations where sampling can be conducted simultaneously (i.e., requiring the same sampling parameters). Thus, the irrigation controller would control the timer to select pre-selected delay and sampling periods, depending on which locations are being sampled, as well as the sequence between the groups.

System 100 includes a plurality of sampling stations 102, of which one is illustrated, and a laboratory station 120. Sampling stations 102 each include one or more extraction tubes 104 implanted in the soil in the location to be monitored. Each extraction tube 104 is coupled by collecting vacuum tubing 106 to a solution collecting bottle 108. Each solution collecting bottle 108, in turn, is coupled via control vacuum tubing 110 to a solenoid valve 114 in a vacuum suction unit 112, of which only a portion is illustrated. Solenoid valves 114 alternately couple vacuum tubing 110 to a vacuum pump (not shown) for providing suction, or to the atmosphere, to release the vacuum. It will be appreciated that, in this embodiment, vacuum is provided substantially all the time, so that no activation signal or reset of the system is required. Rather, the timer can be part of a computerized controller which controls the delay period (if any) and the sampling period.

Vacuum suction unit 112 is coupled or wired to a system suction unit, similar to that described above, which is not shown in the illustration. It will be appreciated that each solution collecting bottle 108 is coupled to its own solenoid valve 114. Typically, each extraction tube 104, even if there is more than one at a sampling station, is coupled to its own collecting bottle.

In the embodiment of FIG. 6, an additional tube 116 is provided for transferring solution from solution collecting bottle 108, at the end of each sampling session, to the lab station 120. Lab station 120 also includes a plurality of solenoid valves 122, each coupling one transfer tube 116 to vacuum tubing 124 leading into an analysis container 126. Solenoid valves 122 have two states—either vacuum or closed. The interior of analysis container is connected by vacuum tubing 128 to a vacuum pump (not shown) for providing suction to introduce, one after the other, solution samples from the various collecting bottles via vacuum tubing 116 and 124 into analysis container 126. A vacuum pipe 130 with associated solenoid valve 132 is provided for emptying analysis container 126, and a fresh water pipe 134 and associated solenoid valve 136 is provided for rinsing or flushing analysis container 126 between solution samples.

A plurality of testing sensors 138 (here represented as a single block) are disposed in analysis container 126 for testing various soil solution parameters, such as salinity, acidity, and concentration of N, P and K, as discussed above, and any other element or compound required by the plants in the location to be monitored, for example, calcium, magnesium, sodium, or even microelements. It will be appreciated that the same tests need not be performed on all the collecting bottles, but that the laboratory station can be programmed to run different tests on samples from different locations or depths, as desired. Preferably, a data connection 140 is provided to permit transfer of the results from the various testing sensors to a data log (not shown), and/or to a central computer or other external location for on-line or off-line analysis of the test results. Data connection 140 can be any conventional data connection, including wired, wireless, and optical.

Operation of the system of the embodiment of FIG. 6 is as follows. Soil sampling is carried out in any of the ways discussed above with reference to FIGS. 1 to 5. The pressure is reduced in vacuum tubing 110 by opening valve 114 in vacuum controller 112 to the vacuum source, and suction of soil solution through extraction tube 104 begins. At this stage, solenoid valve 122 is closed. Once the sampling has ended, the vacuum is released in solution collecting bottle 108, by opening solenoid 114 to the atmosphere, and the analysis container's vacuum pump is activated. Solenoid 122 for a first sampling station is opened, and suction is applied via vacuum tubing 128, 124 and 116 to withdraw the solution collected in solution collecting bottle 108 into analysis container 126. At this time, valves 132 and 136 are closed, while valves 142 and 144 are open.

The collected solution is analyzed by sensors 138, and the data transferred to the data log. Now the analysis container is emptied. Valve 122 is closed, as are valves 136 and 144, valve 142 is opened to the atmosphere, and valve 132 opens vacuum pipe 130 to suction the analyzed solution out of container 126 for disposal. Once container 126 is empty, valve 132 is closed and the procedure begins again with a solution sample from a different collecting bottle in the same or a different sampling station.

If desired, analysis container 126 can be rinsed between each sample. (Rinsing may not be necessary, as in cases where tests are repeated frequently when the solutions are very similar to each other.) This is accomplished by opening valve 136 to permit the inflow of fresh water through fresh water pipe 134 into container 126 to rinse the sensors and the walls of the container. Valves 136 and 144 are then closed, cutting off the flow of fresh water, valve 142 is opened to the atmosphere, and valve 132 is opened to provide vacuum to suction out the rinse water.

It will be appreciated that this embodiment will require that the timer perform additional functions to those described above with reference to FIGS. 1 to 5. First, the timer will be responsible for coordinating operation of the various solenoid valves, to control application and release of the vacuum, introduction of fresh water, and suction removal of solution and rinse water from the analysis container. In addition, the timer will be responsible for coordinating transfer of the collected solutions from the various collecting bottles in the appropriate order and at the appropriate time. If desired, the timer can be part of a computerized controller, which will be programmed to control all these operations.

It is a particular feature of the embodiment of FIG. 6 that the farmer is relieved of the need to manually transport dozens of collecting bottles to a lab at various times of the day in order to receive the results of analysis of the collected solutions.

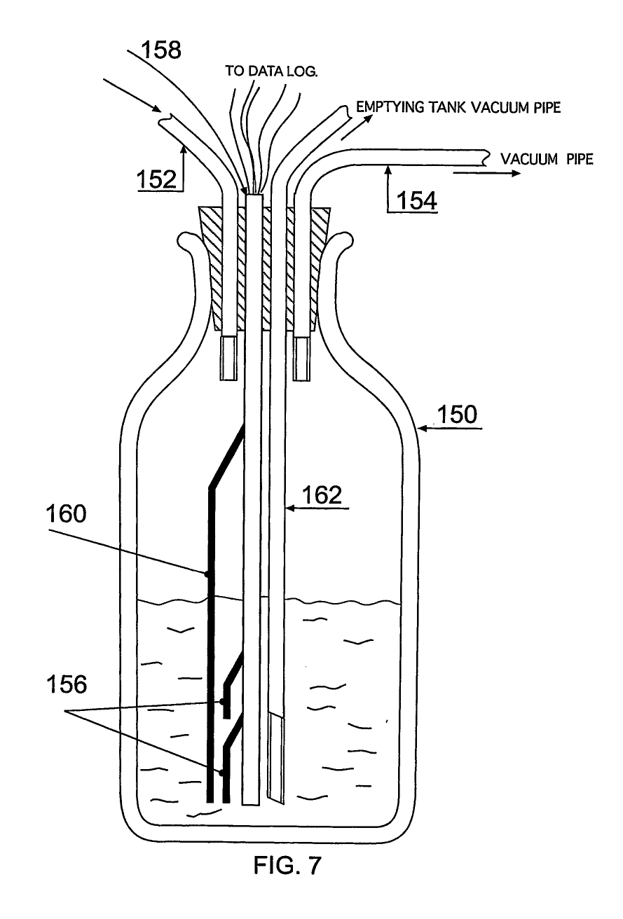

According to an alternative embodiment, illustrated schematically in FIG. 7, the system for collecting soil solution samples can include real-time analysis in each solution collecting bottle, without the need for a central lab at all. FIG. 7 shows a solution collecting bottle 150 according to one embodiment of the invention. Solution collecting bottle 150 includes collecting vacuum tubing 152 coupling the collecting bottle to a sampling device (not shown) implanted in the soil in the location to be monitored. Solution collecting bottle. 150, in turn, is coupled via control vacuum tubing 154 to a vacuum pump (not shown) for providing suction to collect solution from the soil into collecting bottle 150, as described above.

A plurality of testing sensors 156 are disposed in collecting bottle 150 so as to be immersed in the soil solution. Thus, testing sensors 156 are arranged for testing, in real time, various soil solution parameters, such as salinity, acidity, and concentration of N, P and K, and others, such as Ca, Mg, Na, or microelements, as discussed above. Preferably, a data connection 158 is provided to permit transfer of the results from the various sensors to any external system, such as a data log, external lab, and so on. Data connection 158 can be any suitable type of connection. According to one embodiment of the invention, a solution level sensor 160 is also provided inside collecting bottle 150. Solution level sensor 160 can be coupled to the controller of the system (not shown) for providing a signal to indicate the end of the sampling time when a pre-set volume of solution has been collected. Thus, in this case, the solution level sensor would activate the relief valve to stop the vacuum and stop collection of solution, instead of the timer.

A vacuum pipe 162 is provided for emptying collecting bottle 150 between sampling times. If desired, a fresh water pipe and associated solenoid valve can be provided for rinsing out the solution collecting bottle, as described with reference to FIG. 6.

According to one embodiment of the invention, instead of sampling the solution continuously over the sampling period, small samples are withdrawn with a pause between them, without emptying the collecting bottle, so as to monitor the gradual change over the sampling time of various selected parameters of the solution.

One advantage of the embodiments including real-time analysis, such as in FIGS. 6 and 7, is that a warning system can be provided to indicate a soil solution condition which is hazardous to the plant growth. In this case, a database can be provided including the permitted range of each of the elements tested for. When the analyzed data indicates a quantity outside of the permitted range, i.e., excess salinity or acidity, or lack of a crucial element or substance required for plant growth, a warning indication would be provided, audibly, visually, via a message to a cell phone or to a central controller, or in any other fashion which would permit prompt investigation of the situation.

In all the embodiments of the invention, the results of the lab analysis, whether independent of the soil solution sample collection system or part of the system, are provided to the farmer. Other parameters exist, as well. Although they are more difficult to control, mainly in open fields, they are important enough for farmers to take into consideration when making their decisions. These include: day and nighttime outdoor temperatures, the length of daylight, relative humidity, the amount of light and UV radiation (the light spectrum), CO₂ concentration in the air, rainfall, the type and condition of the soil, the type of plant and the plants' stage of growth.

The farmers will evaluate all the information that has been collected, using data tables or graphic curves that show the changes over time for each parameter. The lab analysis, together with information regarding the preferred conditions for each plant at its stage of growth, will be the basis for making short term and long term decisions about fertigation. The farmer can adjust, accordingly, the intervals between irrigation, the amount of water, fertilizer, and the NPK content as elements in the fertigating solution. He or she can also adjust the various sampling parameters, as required, such as the time interval after irrigation, the strength of the vacuum used for suction, the appropriate sampling time, etc.

While the invention has been described with respect to a limited number of embodiments, it will be appreciated that many variations, modifications and other applications of the invention may be made. It will further be appreciated that the invention is not limited to what has been described hereinabove merely by way of example. Rather, the invention is limited solely by the claims which follow.

1. A system for collecting soil solution samples comprising:
a sampling device arranged to be implanted in soil to be monitored, said sampling device being coupled to a vacuum source; and
a suction unit for automatically controlling pressure and time of operation of said vacuum source to provide suction for withdrawing solution from the soil through said sampling device, said suction unit including a relief valve for automatic, abrupt release of vacuum at the end of a pre-defined sampling period.

2. The system according to claim 1, wherein said sampling device is a soil solution extraction tube.

3. The system according to claim 2, further comprising a solution collecting bottle coupled to said soil solution extraction tube by a vacuum tube, wherein said suction unit includes a vacuum pump coupled to said solution collecting bottle to provide suction for withdrawing solution from the soil through said sampling device and into said solution collecting bottle.

4. The system according to claim 1, wherein said suction unit further includes a timer for automatically activating said vacuum pump, and for automatically stopping said vacuum pump and opening said relief valve after a pre-selected sampling period.

5. The system according to claim 4, wherein said timer includes means for providing a pre-selected delay period after termination of irrigation before automatically activating said vacuum pump.

6. The system according to claim 1 wherein the suction unit further comprises:
a vacuum pump controlled by an electric timer switch and a relay switch, connected in series; and
a pressure controller, which also controls the vacuum pump.

7. The system according to claim 6, further comprising reset means for resetting said timer.

8-9. (canceled)

10. The system according to claim 6, further comprising manual activation means for activating said timer.

11. The system according to claim 7, further comprising remote activation means for activating said timer after reset.

12. The system according to claim 1, further comprising a soil moisture sensor coupled to said suction unit for automatically activating said timer to operate said vacuum pump when a pre-selected moisture level is reached in the soil to monitored.

13. A system according to claim 1 comprising:
at least two soil solution extraction tubes arranged to be implanted in soil at different depths; and
a solution collecting bottle coupled to each soil solution extraction tube by a vacuum tube;
wherein said suction unit includes a vacuum pump coupled to each solution collecting bottle for automatically controlling pressure and time of operation of said vacuum pump to provide suction for withdrawing solution from the soil through each said extraction tube and into said coupled solution collecting bottle, said suction unit including a relief valve for automatic, abrupt release of vacuum in each said vacuum tube at the end of a pre-defined sampling period for that extraction tube.

14. The system according to claim 3, further comprising:
a plurality of testing sensors mounted in each solution collecting bottle, said sensors being coupled for communication of sensed data to an external location.

15. The system according to claim 3, further comprising:
a laboratory station selectably couplable to each solution collecting bottle for automatic transfer of solution in said solution collecting bottle to said laboratory station for analysis.

16. The system according to claim 15, wherein said laboratory station includes:
a solution analysis container selectably couplable to each solution collecting bottle in sequence for transfer of said solution from said collecting bottle to said analysis container;
a vacuum suction unit for selectably providing vacuum to each solution collecting bottle for transfer of said solution;
a plurality of testing sensors mounted in said analysis container, said sensors being coupled for communication of sensed data to an external location; and
vacuum pipe for emptying said solution analysis container after testing by said testing sensors.

17. The system according to claim 16, further comprising a fresh water pipe for providing fresh water for rinsing said solution analysis container after emptying.

18. The system according to claim 14, further comprising means for analyzing said sensed data and providing an output corresponding thereto.

19. The system according to claim 18, further comprising a warning system for providing an indication when said sensed data is outside a pre-selected range of data.

20. A method for collecting soil solution samples comprising:
providing vacuum to create suction for withdrawing solution from soil to be monitored;
automatically controlling pressure and time of operation of said vacuum; and
automatically stopping said suction and automatically, abruptly releasing the vacuum at the end of a pre-defined sampling period.

21. The method of claim 20, wherein said step of automatically controlling includes:
activating a timer for counting a time to automatically initiate vacuum to suck solution from soil; and
causing said timer to automatically stop the vacuum and release the vacuum after a pre-selected sampling period

22-27. (canceled)

28. The method according to claim 20, further comprising
automatically transferring solution withdrawn from the soil to a laboratory station for analysis.

29-33. (canceled)
